(19) United States
(12) Patent Application Publication
(10) Pub. No.: US 2010/0073633 A1
(43) Pub. Date: Mar. 25, 2010

(54) FUNDUS CAMERA

(75) Inventors: Koji Uchida, Utsunomiya-shi (JP); Shinya Tanaka, Tokyo (JP); Hiroyuki Inoue, Kawasaki-shi (JP); Hajime Nakajima, Tokyo (JP)

Correspondence Address: CANON U.S.A. INC. INTELLECTUAL PROPERTY DIVISION, 15975 ALTON PARKWAY, IRVINE, CA, 92618-3731, US

(73) Assignee: CANON KABUSHIKI KAISHA, Tokyo (JP)

(21) Appl. No.: 12/565,594

(22) Filed: Sep. 23, 2009

(30) Foreign Application Priority Data
Sep. 25, 2008 (JP) 2008-245861

(51) Int. Cl. A61B 3/14

(52) U.S. Cl. 351/206

(57) ABSTRACT

A fundus camera wherein when a photography start switch is selected, the optical axis of an eye examining section is aligned with the optical axis of an eye by a detection operation of an alignment optical system. After focusing of a fundus image is performed, the eye examining section is moved to a first recording mode position, and a first photograph of the fundus is taken by a stroboscopic tube. Subsequent to taking the first photograph of the fundus, a base length is changed and an alignment position is moved to a second recording mode position so that a second photograph of the fundus is taken.

### BACKGROUND OF THE INVENTION

1. Field of the Invention

The present invention relates to a fundus camera capable of recording a plurality of images and performing a three-dimensional displaying operation.

2. Description of the Related Art

In a related fundus three-dimensional photographing operation, as shown in FIG. 13, an internal fixation target is presented at an eye E to be examined, and two fundus images having different base lengths δ are separately photographed with an image pickup element 1. Then, the two images are arranged horizontally side by side. In addition, using, for example, a stereoscope, the left image is viewed by an examiner with his/her left eye and the right image is viewed by the examiner with his/her right eye. This makes it possible for the examiner to perceive a three-dimensional image of the fundus.

As shown in FIG. 14, two images may be three-dimensionally perceived with eyeglasses 3 by combining the two images with a polarizer 2.

Further, in Japanese Patent Laid-Open No. 6-289355, a special display apparatus that performs a three-dimensional displaying operation by electrical switching between a transparent screen and an opaque screen is required. On the other hand, as discussed in Japanese Patent No. 3929805, a fundus camera that photographs a fundus image by aligning the fundus camera with an eye to be examined using an auto-alignment mechanism is known.

### SUMMARY OF THE INVENTION

The present invention provides a fundus camera that perceives a three-dimensional shape of a fundus by using an auto-alignment mechanism installed for performing an actual fundus photographing operation.

To this end, according to the present invention, there is provided a fundus camera including an illuminating unit configured to illuminate a fundus of an eye to be examined; a viewing photographing optical system configured to form an image of the fundus by receiving light reflected from the fundus due to the illumination by the illuminating unit; an image pickup unit configured to pick up the fundus image formed by the viewing photographing optical system; an index projecting unit configured to project an alignment index onto the eye; an alignment detecting unit configured to calculate information regarding alignment with the eye by receiving a light beam reflected from the alignment index projected by the index projecting unit; and an alignment driving unit configured to align the illuminating unit and the image pickup unit with the eye on the basis of an output of the alignment detecting unit. The alignment driving unit has an ordinary recording mode and a three-dimensional recording mode. In the ordinary recording mode, a still image is recorded by performing alignment with an alignment reference for the eye, and, in the three-dimensional recording mode, a plurality of recordings are made at positions that are displaced by predetermined amounts with respect to the alignment reference in a direction that is horizontal to an optical axis of the viewing projecting optical system.

Other features of the present invention will be apparent from the following description taken in conjunction with the accompanying drawings, in which like reference characters designate the same or similar parts throughout the figures thereof.

### BRIEF DESCRIPTION OF THE DRAWINGS

The accompanying drawings, which are incorporated in and constitute a part of the specification, illustrate embodiments of the invention, and together with the description, serve to explain the principles of the invention.

FIG. 1 is a structural view of an embodiment of the present invention.

FIG. 2 is a structural view of a stop.

FIGS. 3A and 3B illustrate alignment indices.

FIG. 4 is a perspective view of an embodiment of the present invention.

FIG. 5 is a block diagram of the structure of a circuit of a controlling system of a fundus camera.

FIG. 6 is a flowchart for an ordinary recording mode.

FIG. 7 is a flowchart for a still image three-dimensional recording mode.

FIG. 8 is a flowchart for a movie three-dimensional recording mode.

FIG. 9 is a flowchart for a modification of the movie three-dimensional recording mode.

FIG. 10 illustrates a swinging movement.

FIG. 11 illustrates the order of display when taking in horizontally swinging images.

FIG. 12 illustrates the order of display when taking in rotationally swinging images.

FIG. 13 illustrates a photographing operation of stereo images in a related art.

FIG. 14 illustrates perception of stereo images in a related art.

### DESCRIPTION OF THE EMBODIMENTS

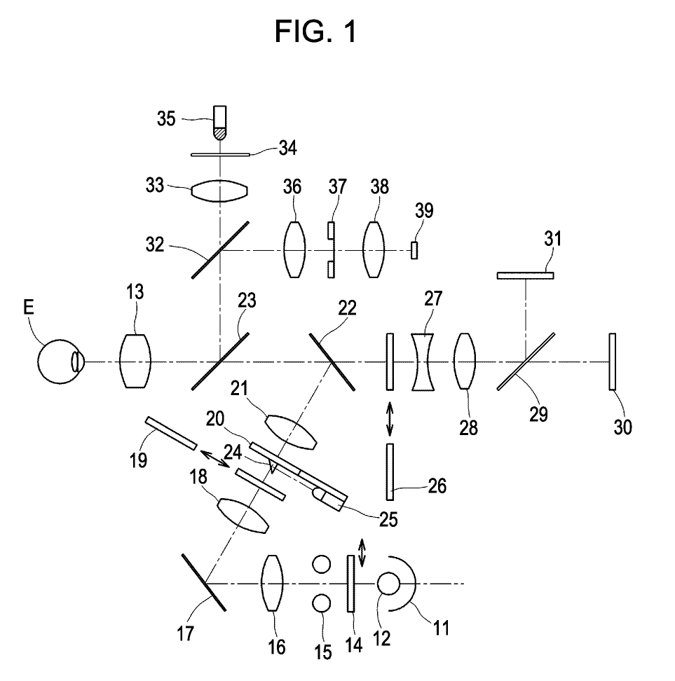

Exemplary embodiments of the present invention will be described in detail in accordance with the accompanying drawings. The present invention will be described in detail with reference to embodiments shown in FIGS. 1 to 12.

FIG. 1 shows a structure including a viewing photographing optical system and an alignment optical system according to a first embodiment. The viewing photographing optical system is provided for illuminating, viewing, and photographing a fundus image. The alignment optical system is provided for detecting the position of an eye E to be examined with respect to a fundus camera.

An illumination optical system is provided between an objective lens 13 and a halogen lamp 12 serving as a viewing light source disposed at the center of a reflecting hemispherical mirror 11. A visible cut filter 14, a stroboscopic tube 15, a lens 16, and a folding mirror 17 are disposed. The visible cut filter 14 can be inserted into and removed from the halogen lamp 12. The stroboscopic tube 15 is a photographic light source. A first relay lens 18, an excitation light filter 19, a split projection unit 20, a second relay lens 21, a mirror 22 having a hole, and a dichroic mirror 23 that reflects infrared light and passes visible light therethrough are successively disposed in a direction of reflection of the folding mirror 17. The split projection unit 20 is provided for a focusing operation. Light of a split index light source 25 exits from the split projection unit 20 through a split prism 24.

The viewing photographing optical system is provided behind the mirror 22 having a hole. In the viewing photographing optical system, a filter 26, a focus lens 27, an imaging lens 28, a quick return mirror 29 that reflects a portion of visible light, and an image pickup unit 30 are successively disposed. In a direction of reflection of the quick return mirror 29, an internal fixation target 31 is disposed. In the internal fixation target 31, LEDs are disposed in a matrix, and a direction of a line of sight of the eye E is set as a guiding index.

An alignment index projection system is disposed in a direction of incidence and a direction of reflection of the dichroic mirror 23. In the alignment index projection system, a dichroic mirror 32, a lens 33, an aperture 34, and an alignment index light source 35 are disposed. The dichroic mirror 32 splits a light beam for projecting an alignment index and a light beam reflected from the eye E of the alignment index.

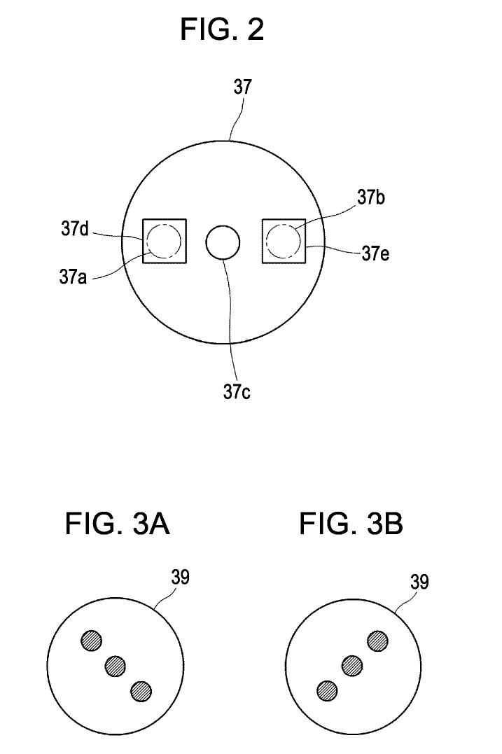

In a direction of reflection of the dichroic mirror 32, an alignment light-receiving optical system is disposed. In the alignment light-receiving optical system, a lens 36, a stop 37, a lens 38, and a CCD sensor 39 are disposed. As shown in FIG. 2, the stop 37 has three openings 37a, 37b, and 37c; and prisms 37d and 37e are disposed at the two outer openings 37a and 37b.

When viewing the fundus of the eye E, the fundus of the eye E is irradiated with light emitted from the halogen lamp 12 through the relay lenses 18 and 21, the mirror 22, and the objective lens 13. Of the viewing light beam transmitted through the objective lens 13, only visible light transmitted through the dichroic mirror 23 passes through the quick return mirror 29 through the focus lens 27 and the imaging lens 28, so that an image is formed on the image pickup unit 30.

The split index light source 25 is turned on, and light exits from the split projection unit 20, so that a split index (provided with visible light having an index of approximately 700 nm (wavelength)) moves along a path similar to that taken by the light from the halogen lamp 12, and is guided to the image pickup unit 30. When the split index is aligned with a proper position by an alignment unit (described later), a split-index displacement amount is detected at the image pickup unit 30. The focus lens 27 is driven so that the split-index displacement amount is eliminated by the alignment detection, to automatically focus a fundus image on the image pickup unit 30.

A light beam emitted from the stroboscopic tube 15 during a photographing operation passes through the excitation filter 19 (inserted in an optical path only for fluorescein fundus angiography and generating excitation light), and annular stroboscopic light reflected by the mirror 22 passes through a pupil of the eye E and illuminates the fundus. A light beam reflected from the fundus of the eye E passes through the hole of the mirror 22, passes through the filter 26 inserted only during the fluorescein fundus angiography, and reaches the image pickup unit 30 provided behind the quick return mirror 29 that is raised out of the optical path.

When performing a mydriatic photographing operation, the visible cut filter 14 is removed from the optical path, and an alignment is performed while viewing the fundus with visible light from the halogen lamp 12. When performing a non-mydriatic photographing operation, the visible cut filter 14 is inserted in the optical path, and the fundus is viewed with infrared light. Therefore, the fundus can be photographed without miosis of a subject and without being too bright.

A light beam emitted from the alignment index light source 35 temporarily forms an image at a turn-around point of the dichroic mirror 23 through the lens 33 and is reflected, and is formed into a parallel light beam by the objective lens 13. The parallel light beam forms an image in an anterior segment of the eye E. A cornea reflection light beam of an alignment index (which is a luminescent spot of the formed image) forms an image at the CCD sensor 39 in the alignment light-receiving optical system. At this time, the image of the anterior segment of the eye E is also formed at the CCD sensor 39 in the alignment light-receiving optical system. The cornea reflection light beam is split into three light beams at the openings 37a to 37c of the stop 37. Luminescent spots that have passed through the two outer prisms 37d and 37e are displaced vertically and form images at the CCD sensor 39 as shown in FIGS. 3A and 3B.

Therefore, when the distance between the eye E and the objective lens 13 deviates from a proper operation distance, the luminescent spots at the CCD sensor 39 become as shown in FIG. 3A. When they come closer to each other, the luminescent spots become as shown in FIG. 3B. Therefore, a front-back direction position of the eye E is known. A positional displacement in an up-down position and a left-right position of the eye E can be detected when all three luminescent stops are as a whole displaced in the up-down direction and the left-right direction on the CCD sensor 39.

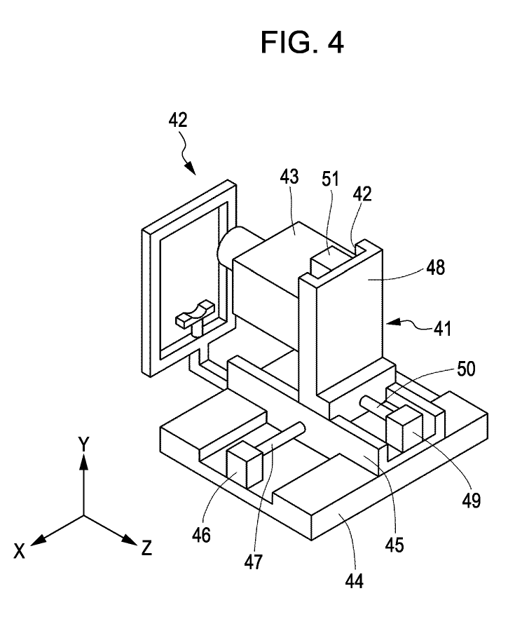

FIG. 4 is a perspective view of the fundus camera having an alignment driving mechanism and including a main body 41 of the apparatus and a subject face securing section 42. The main body 41 of the apparatus includes an eye examining section 43 and three stages that move the eye examining section 43 three-dimensionally in an x-axis direction, a y-axis direction, and a z-axis direction. A groove is formed in the securing section 44 (serving as a base) in the x-axis direction. A movable section 45 is inserted into and fitted to the groove. An internally threaded portion, formed in the movable section 45, engages an externally threaded rod 47 of a driving motor 46 secured to the securing section 44.

Similarly, a groove is formed in the movable section 45 in the z-axis direction. A movable section 48 is inserted into and fitted to the groove. The movable section 48 engages a driving motor 49, secured on the movable section 45, through a threaded rod 50. Further, a groove is formed in the movable section 48 in the y-axis direction. The eye examining section 43 is inserted into and fitted to the groove. The eye examining section 43 engages a driving motor 51, provided at the movable section 48, through a threaded rod (not shown).

These driving motors 46, 49, and 51 are electrically connected to a computation controlling circuit (described later), and can control the eye examining section 43 so as to move to three-dimensional predetermined positions. For the driving motors 46, 49, and 51, pulse motors, DC motors, etc. may be selected. If, as with the rotations of DC motors, the rotations of motors cannot be quantitatively controlled, it is desirable to provide a detecting element in the apparatus for detecting the movement distances of the stages or the amounts of rotations of the driving motors.

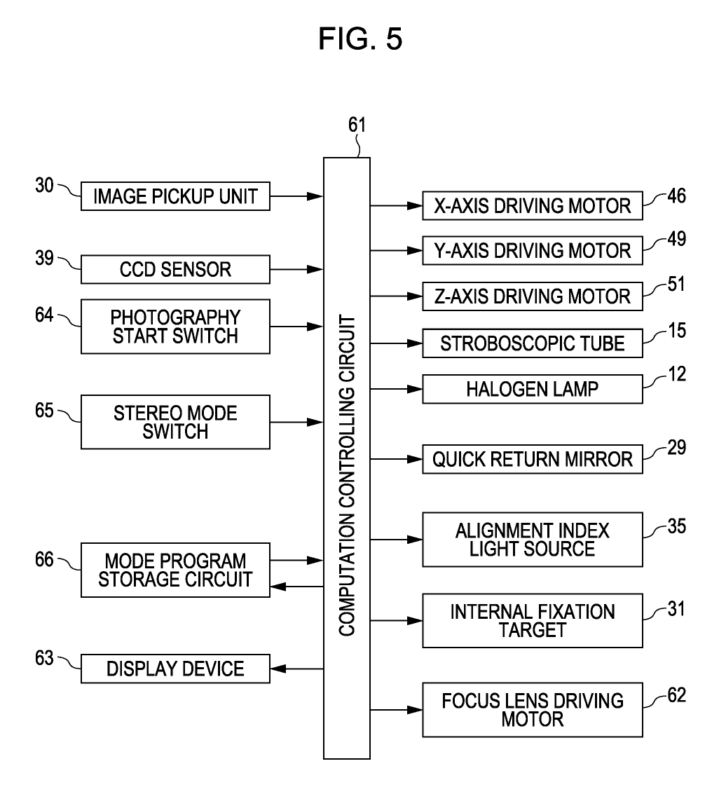

FIG. 5 is a block diagram of the structure of a circuit for performing a motor controlling operation for moving an image pickup element and a photographing section. An output side of a computation controlling circuit 61 is connected to the driving motors 46, 49, and 51; a driving motor 62 that drives the focus lens, the halogen lamp 12; the stroboscopic tube 15; the alignment index light source 35, the internal fixation target 31, the quick return mirror 29, and a display device 63.

Further, output sides of the image pickup unit 30, the CCD sensor 39, a photography start switch 64, a stereo mode switch 65, and a mode program storage circuit 66 are connected to the computation controlling circuit 61.

During auto-alignment, an image signal of an alignment index image obtained by the CCD sensor 39 of the alignment optical system is input to the computation controlling circuit 61. The computation controlling circuit 61 computes a displacement amount of the alignment index image from a predetermined position, and sends a signal to the driving motors 46, 49, and 51 (provided at the stages) so that the alignment index image is at the predetermined position. On the basis of the signal from the computation controlling circuit 61, the driving motors 46, 49, and 51 electrically move the eye examining section 43 in the up-down direction and the left-right direction, so that the eye examining section 43 is aligned at a proper position with respect to the eye E.

In the fundus camera having such a structure, three control patterns of an ordinary recording mode, a still image three-dimensional recording mode, and a movie three-dimensional recording mode are stored in the mode program storage circuit 66.

### Ordinary Recording Mode

An alignment reference position is set so that the eye examining section 43 is aligned in the up-down direction and the left-right direction so as to align the optical axis of the objective lens 13 with the same position as the optical axis of the eye E. In addition, the alignment reference position is set so that the distance between the eye E and the objective lens 13 becomes a proper operation distance.

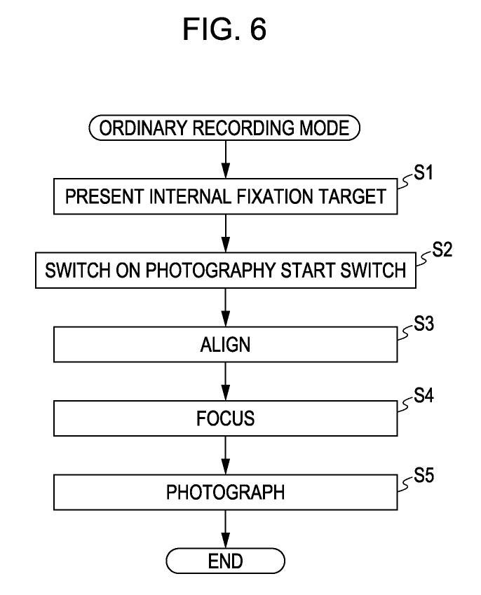

FIG. 6 is a flowchart of operations in the ordinary recording mode. In Step S1, the internal fixation target 31 is presented. When the photography start switch 64 is pressed in Step S2, the optical axis of the eye examining section 43 of the fundus camera is aligned with the optical axis of the eye E in Step S3. When a proper position is reached, focusing of a fundus image is performed in Step S4. One photographing operation is automatically carried out by the stroboscopic tube 15 in Step S5, to obtain a still image in which the center of the eye E is aligned with the center of an image pickup area. The eye opposite to the eye E is guided by the internal fixation target 31. Even if the optical axis of the eye E is tilted by the guiding of the opposite eye, a photographing operation can be performed because the cornea is substantially spherical. In this case, it is possible to photograph an area around the macula lutea and an optic disc, rather than the macula lutea.

### Still Image Three-Dimensional Recording Mode

In the still image three-dimensional recording mode, a total of two still image photographs are taken. In taking the first photograph, the fundus is photographed by the stroboscopic tube 15 in the first recording mode with the optical axis of the objective lens 13 being horizontally displaced by approximately 1 mm towards the left from the optical axis of the eye E. In taking the second photograph after taking the first photograph, the fundus is photographed in the second recording mode with a following second alignment position being displaced horizontally towards the right from the first photograph position by a predetermined amount corresponding to a base length L. The base length L is a length that determines a three-dimensional depth, and may be previously selected from 2 mm, 2.5 mm, and 3 mm and set.

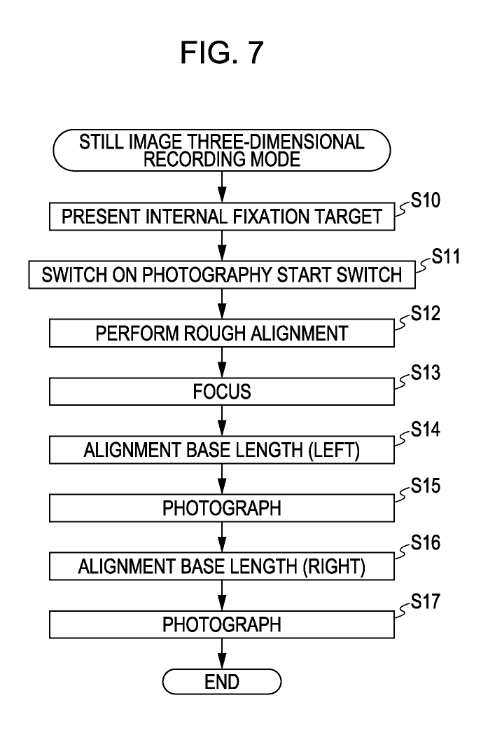

FIG. 7 is a flowchart for the still image three-dimensional recording mode. In Step S10, an examiner moves the position of the internal fixation target 31 so that the eye E faces a portion where three-dimensional recording is to be carried out. When the photography start switch 64 is pressed in Step S11, the optical axis of the eye examining section 43 is aligned with the optical axis of the eye E by a detection operation of the alignment optical system in Step S12. Focusing of a fundus image is performed in Step S13, the eye examining section 43 is moved to a first recording mode position in Step S14, and the fundus is photographed in Step S15. Subsequent to photographing the fundus, the base length L is changed to move the alignment position to a second recording mode position in Step S16, so that the second photograph of the fundus is taken in Step S17.

### Movie Three-Dimensional Recording Mode

In the movie three-dimensional recording mode, after alignment is performed at the position of the first recording mode in the still image three-dimensional recording mode, a movie is photographed while moving it horizontally by an auto-alignment driving mechanism. When the movement speed is 3 mm/s, 25 frames are recorded with a base length of 2.5 mm by recording images at a frame rate of 1/30 seconds. At this time, since the image pickup unit 30 sets a sensitivity that is higher than that in the still image three-dimensional recording mode, a recording operation can be carried out with the light source (that is, the halogen lamp 12), and a strong stroboscopic light does not illuminate the eye E, so that the light is not bright for the subject.

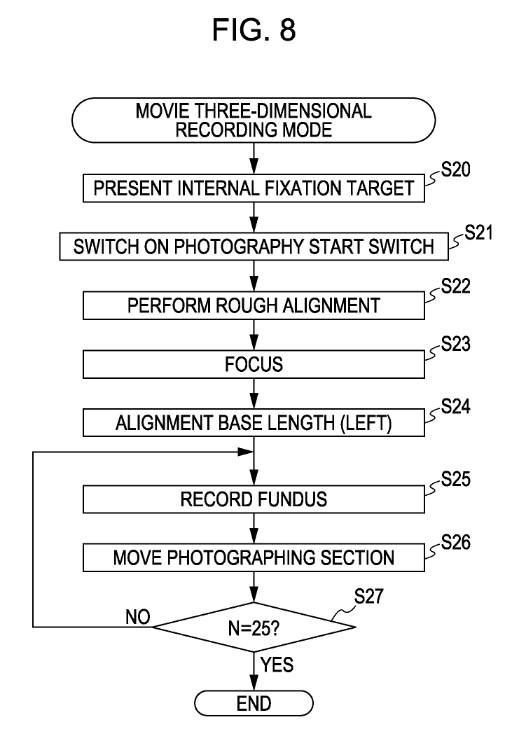

FIG. 8 is a flowchart for the movie three-dimensional recording mode. In Step S20, the examiner moves the position of the internal fixation target 31 so that the eye E faces a portion where three-dimensional recording is to be performed. When the photography start switch 64 is pressed in Step S21, the optical axis of the eye examining section 43 is aligned with the optical axis of the eye E by a detection operation of the alignment optical system in Step S22. Focusing of a fundus image is performed in Step S23, the eye examining section 43 is moved to a first recording position in Step S24, and the fundus is recorded in Step S25. After the recording, the eye examining section 43 is moved horizontally towards the right at the aforementioned speed of 3 mm/s in Step S26, and a processing of taking in and recording images (Step S25) is repeated in Step S27.

By recording the images in this way, three-dimensional recording of movies is carried out for 25 frames over a movement distance of 2.5 mm. By carrying out such a movie recording operation, switching between the base lengths 2 mm, 2.5 mm, and 3 mm in the still image three-dimensional recording mode makes it possible to perform minute recording, thereby it possible to record a larger amount of information in a direction of a three-dimensional depth. In addition, even if three-dimensional eyeglasses or a 3D display is not provided, three-dimensional viewing can be performed by the following displaying method.

For examining, for example, a vein defect of the fundus of the eye E, in a contrast image pickup operation using fluorescent light used for performing a recording operation for a predetermined time after injecting a contrast agent into the vein, it is important to carry out a movie recording operation for a long time as follows. In particular, it is desirable to make it possible to view which of the two blood vessels in a blood-vessel intersecting section is the front side. However, depending upon the eye E, whether a blood vessel exists at a portion that one wants to view cannot be known, thereby making it necessary to perform a recording operation for a certain time after injecting the contrast agent into the vein.

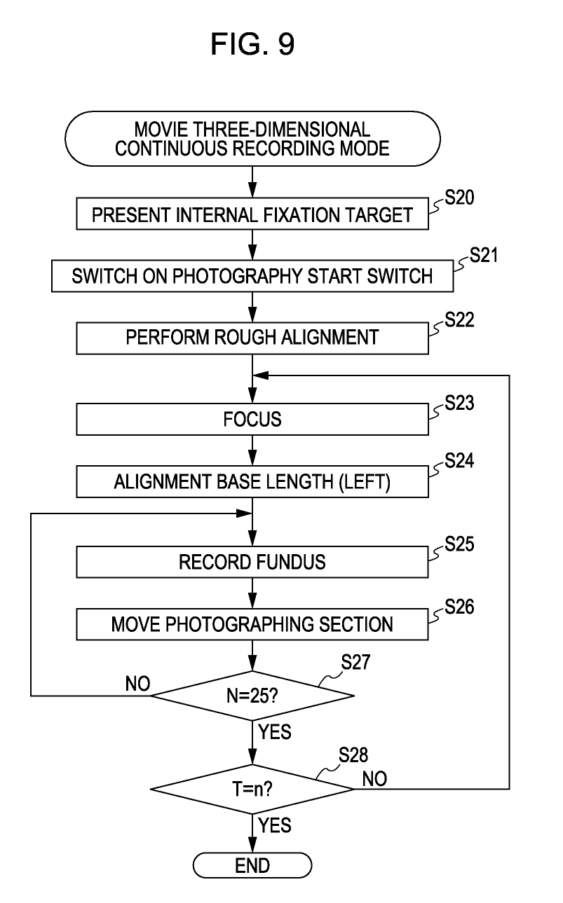

As shown in the flowchart of FIG. 9, when the Step S27 of repeating 25 times the process of recording the fundus (Step S25) and moving the photographing section (Step S26) is controlled so as to be repeated by an n number of times in Step S28, it is possible to record a larger number of fundus movies. Therefore, only particular portions that are recorded are cut after the recording, and a reproduction and a displaying operation are performed. This is useful for examining the blood-vessel intersecting section by a fluorescein fundus angiography described above.

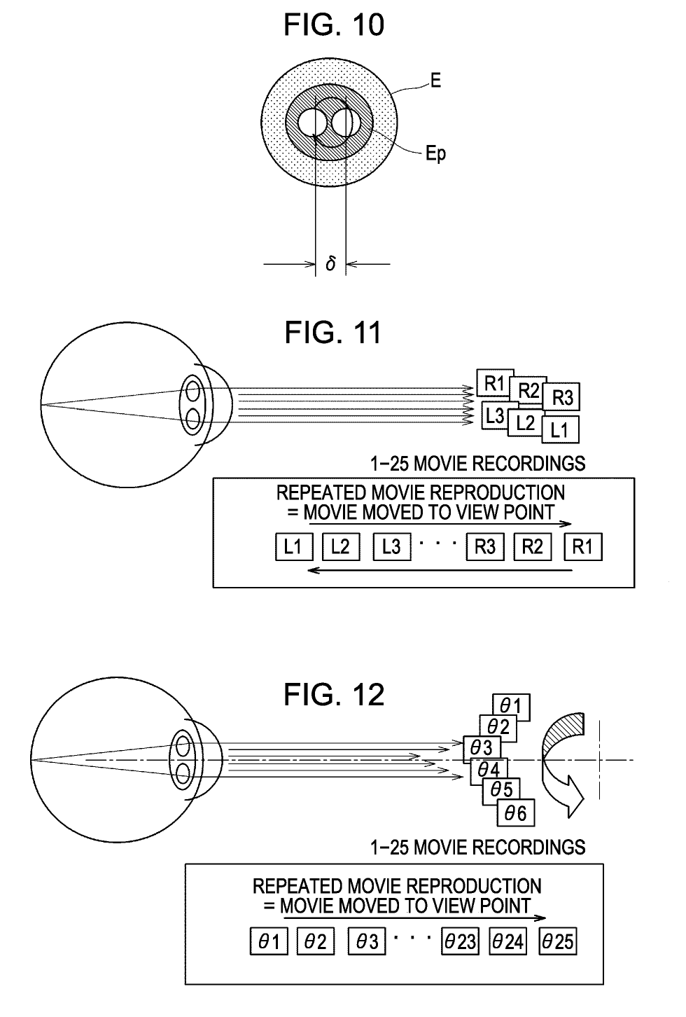

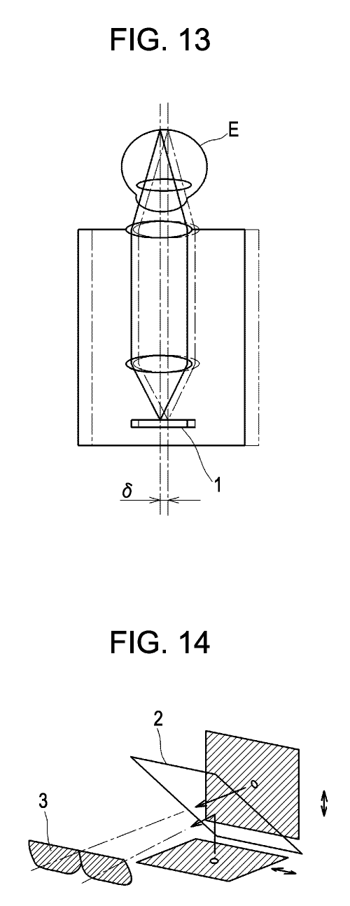

In Step S26, as shown in FIG. 10, it is possible to three-dimensionally view not only the results of a blood vessel that moves vertically, but also the results of a blood vessel that moves horizontally, by causing an alignment reference position to rotationally swing two dimensionally in a pupil Ep in a range of a base length 6. By returning to the Step S23 of performing a focusing operation each time the alignment reference position swings, it is possible to record a focused fundus image in terms of lens adjustment of the eye E. When one eye is successfully photographed in the still image three-dimensional recording mode and the movie three-dimensional recording mode, the alignment reference position is automatically moved to the opposite eye. Accordingly, it is possible to perform both-eye three-dimensional recording in which the opposite eye is similarly photographed.

In the still image three-dimensional recording mode, the display device 63 can display photographed images so that an image situated at the left side of the range of the base length and photographed in the first recording mode and an image situated at the right side of the range of the base length and photographed in the second recording mode can be simultaneously displayed horizontally side by side. In the movie three-dimensional recording mode, when the swinging movement is a horizontal movement, taken-in images are repeatedly and successively reproduced from left to right.

FIG. 11 illustrates the order of display when taking in horizontally swinging images. L1 represents an image taken in at a position at a side closest to a periphery at the left side of the range of the base length, and R1 represents an image taken in at a position at a side closest to a periphery at the right side of the range of the base length. The larger the number, the closer it is to the center of the photographic optical axis. In this case, the display device 63 presents the images as follows: L1→L2→L3, . . . , R3→R2→R1. Starting with the image L1, the images are repeatedly displayed and reproduced at the frame rate. When a viewer is swung towards the left and right, the viewer feels that a three-dimensional depth is provided.

As shown in FIG. 12, by setting the images taken in around the center of the pupil at θ1, θ2, θ3, θ4, . . . , when the images rotating once are continuously reproduced, it is possible to continuously view three-dimensional images horizontally and vertically from various wider angles.

Next, from two or more pairs of images obtained from a swung diagonal position, the computation controlling circuit 61 can calculate a distance D between pixels thereof, a distance fe from a principle point of the eye to the fundus, a photographic magnification m, and a height difference Δ from the base length δ to the fundus:

Δ=fe·D/(δ·m+D)

Therefore, the fundus camera includes an image processing unit that generates a distance image from the distance between the pixels and the photographic magnification m, and the distance to the fundus and the height difference to the fundus. The display device 63 is formed so as to be capable of three-dimensionally displaying an image on the basis of the distance information.

By inserting the visible cut filter (not shown) in the illumination optical system and viewing it using infrared light, non-mydriatic photographing operation in which the eye E does not use a mydriatic agent can be carried out. In the still image three-dimensional recording mode, miosis tends to occur in the second photograph because two photographs are continuously taken with visible light. By setting the internal fixation target so as to guide it slightly towards a peripheral side of the pupil than a photographed section, the peripheral side of the pupil is photographed in the first recording mode, and the center of the pupil is photographed in the second recording mode. This makes it possible to three-dimensionally record still images with little arch factor in which the center becomes dark due to miosis.

While the present invention has been described with reference to exemplary embodiments, it is to be understood that the invention is not limited to the disclosed exemplary embodiments. The scope of the following claims is to be accorded the broadest interpretation so as to encompass all modifications and equivalent structures and functions.

This application claims priority from Japanese Patent Application No. 2008-245861 filed Sep. 25, 2008, the entire contents of which are hereby incorporated by reference herein.

What is claimed is:

1. A fundus camera comprising:
an illuminating unit configured to illuminate a fundus of an eye to be examined;
a viewing photographing optical system configured to form an image of the fundus by receiving light reflected from the fundus due to the illumination by the illuminating unit;
an image pickup unit configured to pick up the fundus image formed by the viewing photographing optical system;
an index projecting unit configured to project an alignment index onto the eye;
an alignment detecting unit configured to calculate information regarding alignment with the eye by receiving a light beam reflected from the alignment index projected by the index projecting unit; and
an alignment driving unit configured to align the illuminating unit and the image pickup unit with the eye on the basis of an output of the alignment detecting unit,
wherein the alignment driving unit has an ordinary recording mode and a three-dimensional recording mode, and
wherein, in the ordinary recording mode, a still image is recorded by performing alignment with an alignment reference for the eye, and, in the three-dimensional recording mode, a plurality of recordings are made at positions that are displaced by predetermined amounts with respect to the alignment reference in a direction that is horizontal to an optical axis of the viewing projecting optical system.

2. The fundus camera according to claim 1, wherein, in the three-dimensional recording mode, a movement of alignment driving unit is a horizontal movement, and
wherein one photograph is taken with a predetermined amount of displacement from a reference position, and then one photograph is taken at the position close to the reference position, as a result of which a total of two still image photographs are taken.

3. The fundus camera according to claim 1, wherein the image pickup unit is capable of performing a movie photographing operation,
wherein the image pickup unit includes an image recording unit that records a movie, and
wherein a movement of the alignment driving unit in the three-dimensional recording mode has a movie three-dimensional recording mode corresponding to a swinging movement.

4. The fundus camera according to claim 3, wherein picking up of the fundus image is performed with a higher sensitivity when recording the movie than when recording the still image.

5. The fundus camera according to claim 3, wherein the alignment driving unit is capable of horizontally and vertically swinging two-dimensionally.

6. The fundus camera according to claim 5, wherein the alignment driving unit applies a rotational motion around the alignment reference as center.

7. The fundus camera according to claim 3, further comprising an image processing unit configured to generate a distance image by calculating parallax of pixels of two or more pairs of images from the two or more pairs of images obtained from diagonal positions swung by the alignment driving unit.

8. The fundus camera according to claim 7, wherein the fundus is capable of being three-dimensionally displayed on the basis of results of the image processing unit.

9. The fundus camera according to claim 1, wherein the illuminating unit includes an excitation-light generating unit configured so that an excitation filter is inserted in an optical path,
wherein the viewing photographing optical system includes an insertion unit configured so that a filter for performing a contrast image pickup operation is inserted in an optical path, and
wherein a movie recording operation by fluorescein fundus angiography is capable of being carried out.

10. The fundus camera according to claim 1, further comprising a displaying unit configured to display a fundus movie image recorded in the three-dimensional recording mode, the displaying unit being capable of repeatedly reproducing the movie image.

11. The fundus camera according to claim 9, wherein the fundus is capable of being three-dimensionally displayed on the basis of results of an image processing unit.
